(19) United States
(12) Patent Application Publication
(10) Pub. No.: US 2023/0081132 A1
(43) Pub. Date: Mar. 16, 2023

(54) TIME-RESOLVED IMMUNOQUANTITATION TEST STRIP FOR DETECTING TETRODOTOXIN IN SHELLFISH FOOD

(71) Applicant: Jiangnan University, Wuxi (CN)

(72) Inventors: Yongli YE, Wuxi (CN); Xiulan SUN, Wuxi (CN); Zhixuan SHI, Wuxi (CN); Jiadi SUN, Wuxi (CN); Jian JI, Wuxi (CN); Yinzhi ZHANG, Wuxi (CN)

(73) Assignee: Jiangnan University, Wuxi (CN)

(21) Appl. No.: 17/884,792

(22) Filed: Aug. 10, 2022

(30) Foreign Application Priority Data
Aug. 10, 2021 (CN) 202110933143.8

(51) Int. Cl. G01N 33/543; G01N 33/558; G01N 33/533

(52) U.S. Cl. CPC G01N 33/54387; G01N 33/558; G01N 33/533

(57) ABSTRACT

The present invention discloses a time-resolved immunoquantitation test strip for detecting tetrodotoxin (TTX) in shellfish food, which belongs to the technical field of rapid detection of time-resolved immunoassay. In the present invention, fluorescent microspheres are adopted to replace the traditional colloidal gold; the fluorescent microsphere is used for labeling a TTX antibody complex; by utilizing a competitive immunization method, the fluorescent microsphere, serving as a fluorescent probe, is used for immunochromatography; and by reading the fluorescence value of a detection line on a fluoroimmunoassay instrument, the TTX in shellfish samples can be analyzed quantitatively and rapidly. The time-resolved immunoquantitation test strip of the present invention can be used for detecting the content of the TTX in various types of shellfish food quantitatively and rapidly and is strong in specificity and high in sensitivity, wherein when the concentration of the TTX is 0.5-40 ng/mL, the logarithmic value of the concentration has a linear relationship with T/To, a linear equation is: Y=0.57365-0.2668LgX, R²=0.9940, and the limit of detection can reach 0.047 ng/mL.

### CROSS-REFERENCE TO RELATED APPLICATIONS

The application claims priority to Chinese patent application No. 202110933143.8, filed on Aug. 10, 2021, the entire contents of which are incorporated herein by reference.

### TECHNICAL FIELD

The present invention relates to a time-resolved immunoquantitation test strip for detecting tetrodotoxin in shellfish food, which belongs to the technical field of rapid detection of time-resolved immunoassay.

### BACKGROUND

Tetrodotoxin (TTX) is one of neurotoxins which are most toxic. The TTX is small-molecule toxic substance of alkaloids. The toxin not only exists in globefishes, but also is widely distributed in various shellfish marine products and phycophyta, such as alexandrium tamarense, nassarius and the like. The TTX is very stable in chemical properties and thermal properties, the TTX cannot be denatured and detoxified at high temperature in a short time, and the structure of the TTX can be completely damaged only after being heated at high temperature for 30 min After entering human bodies, the toxin can block a sodium ion channel on a neural excitation membrane in a manner of high selectivity and high affinity and obstruct neural excitation and conduction in animal bodies, leading to disorder of a regulating function of a central system, so as to cause the phenomena of muscular paralysis, flaccid paralysis, cardiac impulse and so on, finally leading to the failure of a respiratory system and a cardiovascular system. The TTX belongs to a highly toxic substance, and the minimum lethal dose of the human bodies is 0.5 mg. At present, the TTX is detected in various marine organisms and terrestrial organisms, and a lot of TTX poisoning incidents occur in Japan, China, Thailand, Brazil and other countries. In order to control the pollution of the TTX to food, a strict limit standard is formulated. The limit value is explicitly stipulated to be 2.2 mg/kg in Japan. European Food Safety Authority evaluates the risk and supervision of the TTX in bivalves and finally determines that the TTX (less than 44 μg/kg) does not generate the unfavorable effect on humans.

A fluorescence detection method is a first established TTX quantitative method. Additionally, common methods also comprise a mouse detection method, an enzyme-linked immunosorbent assay, a thin layer chromatography, a post-column derivatization HPLC (High Performance Liquid Chromatography) detection method, a GC-MS (Gas Chromatography-Mass Spectrometry) method, an LC-MS (Liquid Chromatography-Mass Spectrometry) method and so on. The TTX is often detected by adopting the GC-MS method and the LC-MS method abroad. GB 5009.206-2016 is the national standard for food safety of the TTX. Although the mouse method and an instrument method are the national standard methods of determining the TTX in the globefishes, the mouse method and the instrument method are detection methods which are complex in operation and need processing of professionals. An immunological detection method is simple in operation and is sensitive and reliable and can be used for detecting various samples of the marine products, which is an ideal detection method.

At present, although test strips for detecting the TTX are reported diversely, such as an immunofluorescence test strip of the Chinese patent CN109444423A, colloidal gold test strips of the patents CN104142394A and CN202522562U, a magnetic sphere test strip of the patent CN108008134A, enzyme-linked immunosorbent assay kits of the patents CN207752014U and CN104133063A and a colloidal gold test strip of the patent CN205982284U, the enzyme-linked immunosorbent assay kit, serving as the only immunodetection in the national standard methods, the detection time is long, and detection equipment such as a microplate reader and the like is larger in volume, so that the enzyme-linked immunosorbent assay kit is not suitable for being used in a site environment; a fluorescent microsphere of the traditional organic fluorescent dye is utilized as a probe, and the traditional organic fluorescent dye is shorter in luminescence lifetime and poor in stability, so that the photobleaching phenomenon occurs easily, and the sensitivity is comparatively lower; and colloidal gold is adopted as an identification probe, the diameter of colloidal gold particles is poor in uniformity, and an immune marker is unstable and can only be used for qualitative detection, so that the content of the TTX cannot be detected rapidly.

In addition, with the rise of temperature, red tides may occur from April to June of every year at the offshore area of China; some algae of the red tides can generate marine algal toxins; and as toxins are accumulated by the toxic red tides, the poisoning incident of consumers occurs after shellfishes are eaten. If the consumers eat the shellfishes (mussels, oysters, clams and so on) polluted by biotoxins, the human bodies may be poisoned seriously. In China, two areas of Putian and Ningde of Fujian, in which the nassarius poisoning incidents occur frequently, are sampled continuously by someone, the residual quantity of marine toxins in samples are analyzed and determined by utilizing the established detection methods, and it is found that the TTX is detected in the nassarius of the two areas. Therefore, establishing a method for detecting the TTX in the shellfishes more sensitively and more rapidly in a manner of big flux becomes an urgent affair.

### SUMMARY

### Technical Problems

An enzyme-linked immunosorbent assay kit, serving as the only immunodetection in national standard methods, the detection time is long, and detection equipment such as a microplate reader and the like is larger in volume, so that the enzyme-linked immunosorbent assay kit is not suitable for being used in a site environment; a fluorescent microsphere of the traditional organic fluorescent dye is utilized as a probe, and the traditional organic fluorescent dye is shorter in luminescence lifetime and poor in stability, so that the photobleaching phenomenon occurs easily, and the sensitivity is comparatively lower; colloidal gold is adopted as an identification probe, the diameter of colloidal gold particles is poor in uniformity, and an immune marker is unstable, so that only qualitative detection can be performed, and the content of TTX cannot be detected rapidly; and at present, a time-resolved fluorescent microsphere test strip for detecting the TTX is still lacked.

### Technical Solutions

In order to solve at least one of the above problems, in the present invention, fluorescent microspheres are adopted to replace the traditional colloidal gold; the fluorescent microsphere is used for labeling a TTX antibody complex; by utilizing a competitive immunization method, the fluorescent microsphere, serving as a fluorescent probe, is used for immunochromatography; and by reading the fluorescence value of a detection line on a fluoroimmunoassay instrument, TTX in shellfish samples can be analyzed quantitatively and rapidly.

The first purpose of the present invention is to provide a time-resolved immunochromatography quantitative test strip for detecting TTX. The test strip comprises a sample pad, a nitrocellulose membrane (an NC membrane) and absorbent paper; a detection line (a T line) and a quality control line (a C line) are arranged on the NC membrane; a TTX complete antigen (TTX-BSA) is coated on the detection line; and a biotin (biotin-BSA) is coated on the quality control line.

In one implementation manner of the present invention, the detection concentration of the TTX is 0.1-1000 ng/mL.

In one implementation manner of the present invention, the dosage of the TTX complete antigen is 0.3-8 mg/mL, and the dosage of the biotin is 0.1-1 mg/mL.

In one implementation manner of the present invention, the distance between the detection line and the quality control line is 4-6 mm.

In one implementation manner of the present invention, the width of the time-resolved immunochromatography quantitative test strip is 3-5 mm.

In one implementation manner of the present invention, for the detection line, the 0.3-8 mg/mL TTX complete antigen is sprayed on the NC membrane, and the spraying quantity per centimeter of the width of the detection line on the NC membrane is 1-2 μL; and when the width of the NC membrane is 0.4 cm, the spraying quantity is 0.4-0.8 μL.

In one implementation manner of the present invention, for the quality control line, the 0.1-1 mg/mL biotin is sprayed on the NC membrane, and the spraying quantity per centimeter of the width of the quality control line on the NC membrane is 1-2 μL/cm. When the width of the NC membrane is 0.4 cm, the spraying quantity is 0.4-0.8 μL.

In one implementation manner of the present invention, the sprayed NC membrane is placed in a vacuum drying oven at 37° C. and is dried for standby application.

In one implementation manner of the present invention, the TTX complete antigen is diluted to the final concentration of 0.3-8 mg/mL by 0.01 M of phosphate buffer solution (PBS) with the pH value of 7.4.

In one implementation manner of the present invention, the biotin is diluted to the final concentration of 0.1-1 mg/mL by 0.01 M of phosphate buffer solution with the pH value of 7.4.

In one implementation manner of the present invention, in the time-resolved immunochromatography quantitative test strip, the absorbent paper, the NC membrane and the sample pad are adjacent sequentially, and the length of the overlapping area of adjacent parts is 2-4 mm; and an overlapping part of the absorbent paper and the NC membrane and an overlapping part of the sample pad and the NC membrane are respectively located on the upper side of the NC membrane.

In one implementation manner of the present invention, in a preparation method of the TTX complete antigen (TTX-BSA), the TTX complete antigen (TTX-BSA) is obtained by coupling a TTX standard substance and a carrier protein; and the preparation method specifically comprises the following steps:

dissolving 0.5 mg of TTX in 1 mL of ultrapure water to obtain TTX solution; dissolving BSA in 2.0 mL of PBS (pH7.4), then adding the TTX solution and uniformly mixing the mixture, so as to obtain mixed solution; adding 37% formaldehyde solution dropwise into the mixed solution until the final volume (the mixed solution+ formaldehyde) fraction is 1.5%, and uniformly mixing the mixture, so as to obtain reaction solution; slowly shaking the reaction solution for water bath at 30° C. for reacting for 72 h, dialyzing for 48 h at 4° C., and removing residual free TTX through the changes of 1 L of PBS (pH7.4) of six times; and obtaining a TTX-BSA conjugate, i.e., the TTX complete antigen, and preserving TTX-BSA conjugate at−20° C.

The second purpose of the present invention is to provide a method of detecting TTX in shellfishes by a time-resolved immunoquantitation test strip, which comprises the following steps:

(1) extraction of a to-be-detected substance: homogenizing shellfish meat, heating, cooling, centrifuging, and collecting supernate; and then, degreasing to obtain the to-be-detected substance;

(2) uniformly mixing a Eu³⁺-fluorescent microsphere labeled TTX monoclonal antibody Eu-TTX-mAb, a Eu³⁺-fluorescent microsphere labeled streptavidin Eu-SA, the to-be-detected substance and buffer solution for hatching, so as to obtain to-be-detected solution;

(3) adding the to-be-detected solution obtained in step (2) into the sample pad in the time-resolved immunochromatography quantitative test strip described in the present invention, and then determining the fluorescence intensity value at the detection line of a sample: a T value; and

(4) substituting the obtained T value into a quantitative curve, so as to obtain the concentration of the TTX in the to-be-detected solution.

In one implementation manner of the present invention, in the extraction of the to-be-detected substance in step (1), the extraction is performed according to GB/T5009, which comprises the following steps:

collecting 5 g of the shellfish meat, adding 25 mL of 0.1% glacial acetic acid solution for homogenizing, and performing water bath at 100° C. for 10 min after homogenizing; standing for cooling, centrifuging at 10,000 r/min for 5 min, collecting supernate, adding an appropriate amount of 0.1% glacial acetic acid into precipitates, uniformly mixing the mixture and performing water bath for 5 min; standing for cooling, centrifuging at 10,000 r/min for 5 min, collecting supernate, precipitating and repeating the above step once; collecting the supernate three times to the constant volume of 50 mL; then, adding 5 mL of chloroform and degreasing for 30 min, during which shaking for uniform mixing 4-6 times, centrifuging at 10,000 r/min for 3 min, separating an aqueous phase and chloroform, collecting the aqueous phase, degreasing again once, and adjusting the pH value of the aqueous phase to 7.0 with NaOH, so as to obtain the to-be-detected substance; and if the to-be-detected substance is not detected directly, preserving the to-be-detected substance at 4° C. (the pH value does not need to be adjusted) (GB/T5009).

In one implementation manner of the present invention, the volume ratio of the Eu³⁺ -fluorescent microsphere labeled TTX monoclonal antibody Eu-TTX-mAb, the Eu′-fluorescent microsphere labeled streptavidin Eu-SA, the to-be-detected substance and the buffer solution in step (2) is 2:10:45:35.

In one implementation manner of the present invention, a Eu³⁺-fluorescent microsphere in step (2) is obtained in a manner that a lot of Eu³⁺ fluorescence chelated is wrapped in a nanoscale polystyrene microsphere; and a carboxylated group is modified on the surface of the polystyrene microsphere and is used for coupling the protein, and the fluorescent marking efficiency of the protein (the antigen/the antibody) is improved, so as to effectively improve the sensitivity of analysis.

In one implementation manner of the present invention, the particle diameter of the Eu³⁺-fluorescent microsphere in step (2) is 100-300 nm; the Eu³⁺-fluorescent microspheres are usually dispersed in the buffer solution for use; the solid content in the Eu³⁺-fluorescent microsphere solution is 1%-10%; and the percentage is the mass percentage.

In one implementation manner of the present invention, the Eu³⁺-fluorescent microsphere labeled TTX monoclonal antibody Eu-TTX-mAb in step (2) is prepared by combining the EDC (Dichloroethane) and NHS (N-Hydroxyl Succinimide) mediated activation and an amino of an antibody molecule to form an amido bond.

In one implementation manner of the present invention, the hatching in step (2) is carried out at 20-30° C. for 10-20 min.

In one implementation manner of the present invention, the adding amount of the to-be-detected solution in step (3) is 80-95 μL.

In one implementation manner of the present invention, the chromatography in step (3) is carried out at 37° C. for 15-20 min.

In one implementation manner of the present invention, for the determination of the fluorescence intensity value in step (3), the fluorescence intensity value is tested by using an HG-98 immunoquantitation analyzer.

In one implementation manner of the present invention, the quantitative curve in step (4) is Y=0.57365-0.2668LgX, R²=0.9940, IC50=1.713 ng/mL, and the limit of detection is 0.047 ng/mL, wherein Y represents T/To, and X represents the concentration of the TTX.

The third purpose of the present invention is application of the method of the present invention in the field of food detection.

The fourth purpose of the present invention is application of the time-resolved immunochromatography quantitative test strip of the present invention in the field of food detection.

[Beneficial Effects]

(1) The method of the present invention can be used for detecting the content of the TTX in various types of shellfish food quantitatively and rapidly and is strong in specificity and high in sensitivity, wherein when the concentration of the TTX is 0.5-40 ng/mL, the logarithmic value of the concentration has a linear relationship with T/To, a linear equation is: Y=0.57365-0.2668LgX, R²=0.9940, and the limit of detection can reach 0.047 ng/mL. The detection method provided by the present invention is wide in detection range, and the linear range of quantification can reach 0.5-40 ng/mL. The detection method is stable and reliable, the sample addition recovery rate is 97.07%-101.48%, and the variable coefficients are less than 4.70%.

(2) In the method of the present invention, the TTX monoclonal antibody is taken as an identification target spot, the time-resolved fluorescent microspheres are taken as signal sources, and the volume of the fluorescent microsphere is much larger than the volume of a fluorescent dye molecule, so that the non-specific fluorescence interference can be effectively eliminated, the particle diameters are uniform, and the particle diameter is increased to 300-400 nm after the fluorescent microsphere is connected with the TTX-mAb, which indicates that the fluorescent microspheres and the monoclonal antibody form a novel complex.

(3) The test strip of the present invention is high in sensitivity, low in cost, simple and convenient in operation, rapid and quantitative in detection and good in stability, is suitable for being used in industry and commerce departments, third-party detection institutions, governmental supervision departments at all levels, shellfish food enterprises and so on, is wide in market prospect and is easy to be popularized and used.

### BRIEF DESCRIPTION OF DRAWINGS

FIG. 1 is a transmission electron micrograph of a tetrodotoxin (TTX) antibody coupling to a fluorescent microsphere, wherein A is an electron micrograph of the antibody coupling to the fluorescent microsphere at a low magnification; and B is an electron micrograph of the antibody coupling to the fluorescent microsphere at a high magnification.

FIG. 2 is a diagram showing ultraviolet verification on a TTX complete antigen.

FIG. 3 is a structural diagram of an immune test stripe for TTX.

FIG. 4 is a diagram showing a standard curve A and a quantitative curve B of detecting TTX with a labeling fluorescent microsphere immunochromatographic assay and a test strip diagram C under an ultraviolet lamp.

FIG. 5 is a diagram showing optimization on concentration of a complete antigen of a detection line (T line) with TTX immunochromatographic assay.

FIG. 6 is a diagram showing T line immunodynamic curves of TTX immunochromatographic assay, wherein with fluorescence intensity values and T/C as ordinates and reaction times as abscissas, an immunoreaction kinetic curve (A) is drawn; and (B) shows changes on fluorescence intensity value at different immunoreaction times.

FIG. 7 is a diagram showing a standard curve A and a quantitative curve B of detecting TTX in shellfish.

FIG. 8 is a diagram showing a TTX specificity test.

### DETAILED DESCRIPTION OF THE EMBODIMENTS

The preferred embodiments of the present invention are described below. It should be understood that the embodiments are for better explaining the present invention, but not used for limiting the present invention. In the embodiments, used streptavidin-fluorescent microspheres are purchased from Suzhou VDO Biotech Co., Ltd. with a solid content of 1% and are used after being diluted by 500 folds by a self-made microsphere reconstituted solution, wherein the self-made microsphere reconstituted solution is 0.05 M Tris-HCl containing 5% sucrose, 1% bovine serum albumin and 1% Tween-20 and having a pH value of 7.0.

### Embodiment 1 Preparation of Tetrodotoxin Complete Antigen (TTX-BSA)

1. Preparation of tetrodotoxin complete antigen (TTX-BSA)

A TTX standard substance was coupled to bovine serum albumin (BSA) to obtain an artificial antigen

0.5 mg of TTX was taken and dissolved into 1 mL of ultrapure water, and a mixture was stirred for clarification, denoted as a reaction liquid A; 4 mg of BSA was weighed and dissolved into 2.0 mL of PBS (pH 7.4) to obtain a protein solution; the reaction liquid A was slowly added to the protein solution dropwise for uniform mixing, denoted as a reaction liquid B; 37% formaldehyde solution was slowly added to the reaction liquid B dropwise until a final volume fraction was 1.5%, and uniform mixing was performed; then, a mixed solution was slowly shaken in a water bath at 30° C. for reacting for 72 h and was dialyzed for 2 d at 4° C. by using 0.01 M phosphate buffer solution (PBS) with changing dialysate for 3 times every day, so as to remove residual free TTX; and a TTX-BSA conjugate was obtained, i.e., the TTX complete antigen (TTX-BSA), and was preserved at −20° C.

2. Identification of TTX antibody

The TTX standard substance, a carrier protein and the TTX complete antigen were subjected to ultraviolet (200-300 nm) scanning determination for comparing differences among the three.

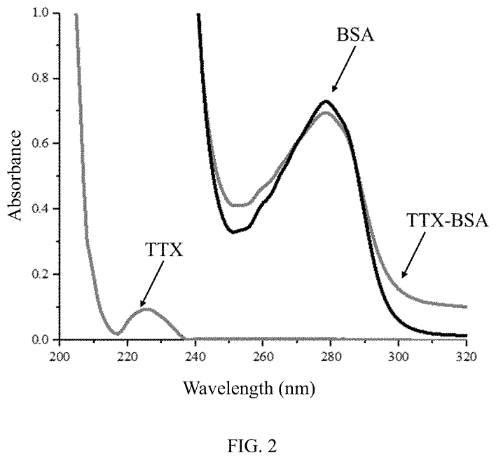

A result is shown in FIG. 2. From FIG. 2: compared with spectrum shapes or peak values of the TTX standard substance and the carrier protein, a spectrum shape or a peak value of the TTX complete antigen have an obvious change, which shows that synthesis of the TTX complete antigen is successful.

### Embodiment 2 Preparation of Fluorescent Probe for Eu³⁺-Fluorescent Microsphere Labeled TTX Monoclonal Antibody

Preparation of Relevant Solution:

Activation buffer: 0.05 M 2-(N-morpholino) ethanesulfonic acid (MES, C₆H₁₃NO₄SH₂O) solution with a pH value of 6.0;

coupling buffer: 0.01 M phosphate buffer solution (PBS) with a pH value of 7.0 (a solvent with free amine is avoided from being used);

activator: 2 mg/mL 1-(3-dimethylaminopropyl)-3-ethylcarbodiimide hydrochloride (EDC, C₈H₁₇N₃HC₁) solution and 2 mg/mL N-hydroxysuccinimide (NHS, C₄H₅NO₃) solution; blocking buffer: 0.01 M phosphate buffer solution (PBS) containing 1% BSA and 0.05% Tween-20 and having a pH value of 6.0;

antibody basic protective liquid: 0.05 M Tris-HCl containing 5% trehalose, 0.1% PVPK-30, 0.5% BSA and 0.1% TritonX-100 and having a pH value of 7.0;

label washing liquid: 0.05 M Tris-HCl containing 0.1% Tween-20 and having a pH value of 7.0; and

microsphere reconstituted solution/reaction slow-release liquid: 0.05M Tris-HCl containing 5% sucrose, 1% bovine serum albumin and 1% Tween-20 and having a pH value of 7.0.

A specific preparation method for Eu-TTX-mAb comprises the following steps:

(1) 10 μL of fluorescent microsphere solution wrapping Eu′ therein and modified with a carboxyl functional group (purchased from Taizhou Bionano New Material Science Co., Ltd. and having a particle diameter of 300 nm) (having a solid content of 1%) placed at 4° C. was taken for ultrasonic dispersion, the activation buffer with a concentration of 900 μL was added for centrifugation for 5 min at 15000 rpm and 4° C.

(2) A supernate was discarded, 600 μL of activation buffer was added for ultrasonic resuspension, and centrifugal cleaning was repeated for 3 times.

(3) A supernate was discarded, 200 μL of activation buffer was added for ultrasonic resuspension, 5 μL of 2 mg/mL EDC solution and 5 μL of 2 mg/mL NHS solution were added as the activator, and oscillation was performed in a shaker in the dark for 30 min at a room temperature and 500 rpm.

(4) After activation was completed, centrifugation was performed; a supernate was discarded; and the PBS was added for cleaning for 3 times.

(5) A supernate was discarded, 400 μL of coupling buffer was added for ultrasonic resuspension, then 10 μg of TTX monoclonal antibody (references for a preparation method: ([1] Lei Cong. Preparation of Tetrodotoxin Specific Monoclonal Antibody [D]. Shanghai Ocean University, 2011)) was added, and oscillation labeling was performed for 3 h in the dark at the room temperature.

(6) After labeling was completed, 10% (v/v) blocking buffer and 100 μL of antibody basic protective liquid were added, and oscillation was performed for 40 min in the dark at the room temperature.

(7) After blocking, centrifugation was performed to discard a supernate, a resultant was cleaned for 3 times with 900 μL of label washing liquid.

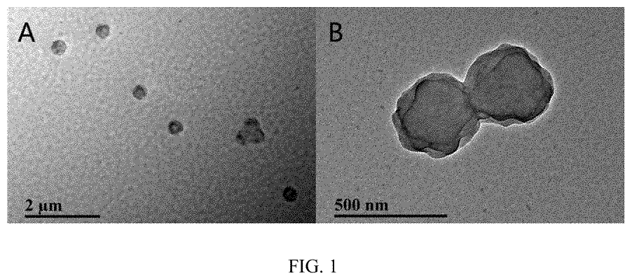

(8) A supernate was discarded, 200 μL of fluorescent microsphere reconstituted solution was added, and a Eu³⁺-fluorescent microsphere labeled TTX monoclonal antibody Eu-TTX-mAb (a transmission electron micrograph shown as FIG. 1) was obtained and preserved at 4° C. for standby application.

From FIG. 1: the Eu-TTX-mAb is stable and controllable in surface functional group, good in experiment repetition, strong in detection specificity, capable of achieving rapid quantitative detection, high in sensitivity and small in error and thus provides great convenience and rapidness for immediate detection.

### Embodiment 3 Preparation of TTX Immune Test Stripe Based on Labeling Fluorescent Microsphere

A method of preparing a time-resolved immunochromatography quantitative test strip for detecting TTX, comprising the following steps:

(1) A tetrodotoxin complete antigen (TTX-BSA) solution (0.4 mg/mL) and a biotin solution (0.3 mg/mL) were sprayed on a nitrocellulose membrane (NC membrane) with the spraying amount in each of 1 μL/cm (an instrument parameter, referring to that 1 μL of to corresponding solution was sprayed on the NC membrane once a nozzle moves by 1 cm every time) to serve as a detection line (T line) and a quality control line (C line). Widths of the T line and the C line depend on a diameter of a pipeline of a membrane spraying instrument and are 2 mm; a distance between the T line and the C line is about 5 mm; and the T line and the C line are dried for 3 h at 37° C. An XYZ3050 model three-dimensional dispensing platform from American BioDot Inc. is employed for spraying.

A sample pad, the nitrocellulose membrane and absorbent paper were adhered to a PVC basal plate to assemble a test strip. The key for assembly of the test strip is to guarantee consistent transitivity among various parts, wherein the T line is distant from the sample pad by about 5 cm; the C line is distant from the absorbent paper by about 5 cm; the sample pad overlaps on the nitrocellulose membrane with an overlap of about 3 mm; and similarly, the absorbent paper overlaps on the nitrocellulose membrane with an overlap of about 3 mm.

The plate subjected to adhesion was cut into the test strip with a width of about 4 mm by a strip cutter, the test strip was assembled by using a plastic base and a clamping housing to obtain an immunochromatography quantitative test strip, and the immunochromatography quantitative test strip was sealed and preserved at 4° C. for standby application.

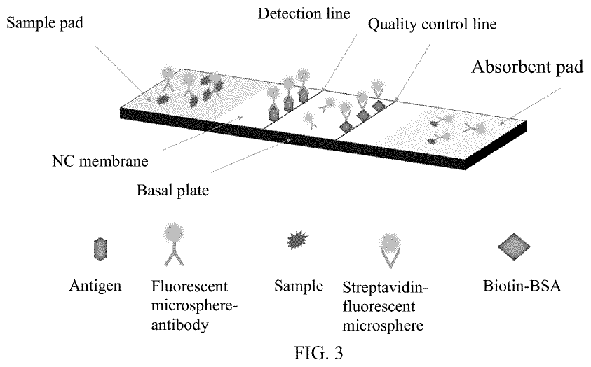

A structural diagram of the immunochromatography quantitative test strip is shown as FIG. 3. The sample pad, the nitrocellulose membrane and the absorbent paper are sequentially provided on the basal plate from left to right.

### Embodiment 4 Drawing of Standard Curve of Tetrodotoxin Immunochromatography Quantitative Test Strip

A drawing method of a standard curve comprises the following steps:

A TTX standard substance was taken and added to a phosphate buffer solution (pH 7.0,0.01M) to prepare standard solutions with concentrations of TTX of 0 ng/mL, 0.1 ng/mL, 0.2 ng/mL, 0.5 ng/mL, 1 ng/mL, 4 ng/mL, 8 ng/mL, 20 ng/mL, 40 ng/mL, 100 ng/mL, 200 ng/mL, 800 ng/mL and 1000 ng/mL respectively; and the standard solutions were used for fluorescent test strip detection.

The Eu³⁺-fluorescent microsphere labeled TTX monoclonal antibody (Eu-TTX-mAb) prepared in embodiment 2 served as a fluorescent probe; 45 μL of standard solution, 2 μL of fluorescent probe (Eu-TTX-mAb solution: 0.05 μg/μL fluorescent microsphere labeled TTX monoclonal antibody, 0.1 M Tris-HCl solution containing 5% sucrose, 1% bovine serum albumin and 1% Tween-20 and having a pH value of 7.5), 10 μL of streptavidin-fluorescent microsphere and 35 μL of standard substance buffer (the standard substance buffer is 0.01 M PBS (pH 7.4) containing 5% methanol (v:v)) were uniformly mixed; and a mixture was hatched for 10 min at a room temperature to obtain 92 μL of to-be-detected solution.

Then, 92 μL of to-be-detected solution was slowly added to the sample pad of the immunochromatography quantitative test strip in embodiment 3 dropwise for chromatography for 20 min at 37° C.; a fluorescence value of the T line on the test strip was recorded by using an HG-98 immunoquantitation analyzer with 6 replicates tested for each concentration; and setting a fluorescence value of the T line of the standard solution with the concentration of 0 ng/mL as T₀ and fluorescence values of the T lines of the standard solutions with other concentrations as T, the standard curve was drawn with a logarithmic value of the concentration of each TTX standard substance as an abscissa and T/T₀×100 (%) as an ordinate, wherein a competitive inhibition rate was set as (1-T/T₀)×100%.

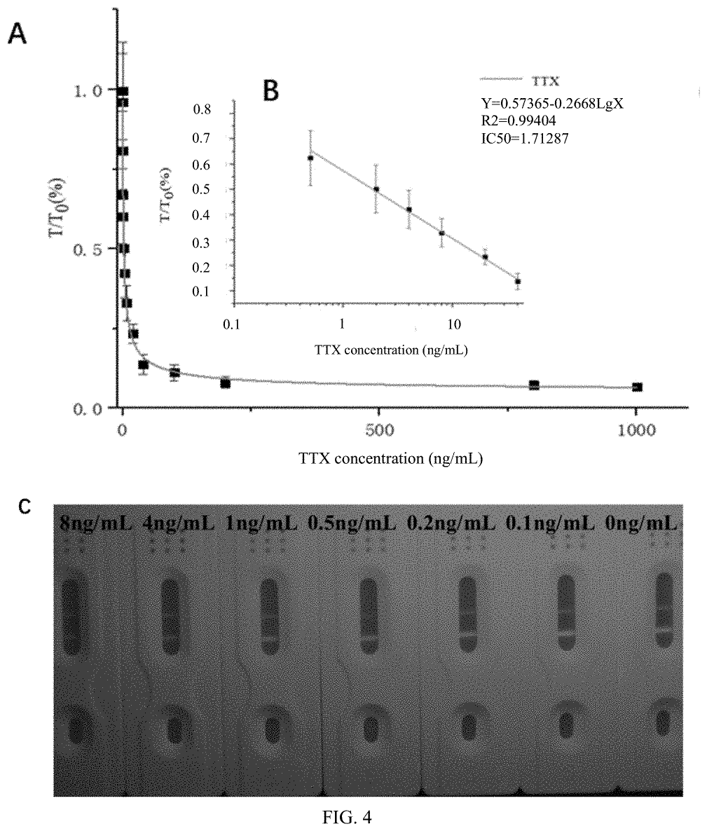

From A in FIG. 4, with the increase of the concentration of the TTX, fluorescence on a band of the T line of the test strip would become lighter and lighter, so T/To would become smaller and smaller. As shown in FIG. 4, B is a change curve (quantitative curve) of T/To along with the concentration of TTX; when the concentration of the TTX is 0.5-40 ng/mL, the logarithmic value of the concentration of the TTX has a linear relationship with T/T0; a linear equation is: Y=0.57365-0.2668LgX, R²=0.9940, and IC50=1.713 ng/mL; and a limit of detection is 0.047 ng/mL.

### Embodiment 5

A method of detecting tetrodotoxin in shellfishes by a time-resolved immunoquantitation test strip, comprising the following steps:

(1) extraction of a to-be-detected substance:

5 g of shellfish meat was collected, 25 mL of 0.1% glacial acetic acid solution was added for homogenizing, and a water bath was performed at 100° C. for 10 min after homogenizing; standing was performed for cooling, centrifugation was performed at 10,000 r/min for 5 min, a supernate was collected, an appropriate amount of 0.1% glacial acetic acid solution was added to precipitates for uniform mixing, and a water bath was performed for 5 min; standing was performed for cooling, centrifugation was performed at 10,000 r/min for 5 min, a supernate was collected, and the above step was repeated for precipitates once; the three supernates were collected to a constant volume of 50 mL; then, 5 mL of chloroform was added for degreasing for 30 min, during which shaking was performed for uniform mixing 5 times, centrifugation was performed at 10,000 r/min for 3 min to separate an aqueous phase and chloroform, an aqueous phase was collected, degreasing was performed once again, and a pH value of the aqueous phase was adjusted to 7.0 with NaOH, so as to obtain the to-be-detected substance; and if not being detected directly, the to-be-detected substance might be preserved at 4° C. (without adjusting the pH value) (GB/T5009).

(2) 2 μL of Eu³⁺-fluorescent microsphere labeled TTX monoclonal antibody Eu-TTX-mAb, 10 μLof Eu³⁺-fluorescent microsphere labeled streptavidin Eu-SA, 45 μL of to-be-detected substance and 35 μL of buffer solution were uniformly mixed for hatching for 10 min at a room temperature, so as to obtain 92 μL of to-be-detected solution.

(3) The to-be-detected solution obtained in step (2) was added to the sample pad in the time-resolved immunochromatography quantitative test strip described in embodiment 3 for chromatography for 20 min at 37° C., and then a fluorescence intensity value (: a T value) at the detection line of the to-be-detected solution was recorded by the HG-98 immunoquantitation analyzer.

(4) The obtained T value was substituted into the quantitative curve in embodiment 4, so as to obtain the concentration of the TTX in the to-be-detected solution.

### Embodiment 6 Condition Optimization

(1) Optimization of tetrodotoxin complete antigen

In order to improve the detection sensitivity of a time-resolved immunoquantitation test strip, the effect of different dilution concentrations of the TTX complete antigens on a competitive inhibition rate between a negative control group and a sample with the TTX content of 0.25 μg/L in the experimental process is researched.

The specific experimental exploration process is as follows:

the concentration of a TTX complete antigen (TTX-BSA) solution sprayed at a detection line T in embodiment 3 was adjusted; specifically, dilution folds of the TTX-BSA were 5, 10, 15, 20 and 25, concentrations were specifically 1.6 mg/mL, 0.8 mg/mL, 0.5 mg/mL, 0.4 mg/mL and 0.3 mg/mL, and other kept consistent to those in embodiment 3; and then the time-resolved immunoquantitation test strips for different concentrations of the TTX complete antigen were obtained.

The negative control group (5% methanol-PBS) and a positive test group (with the TTX concentration of 0.25 μg/L) were analyzed, and then the effect of different concentrations of the TTX-BSA on a detection method was evaluated.

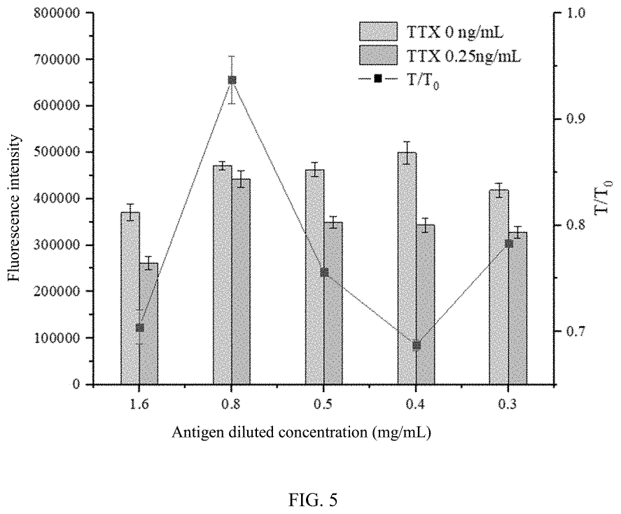

From FIG. 5, with the increase in the concentration of the TTX-BSA at the T line, the fluorescence intensity between the negative control group (5% methanol-PBS) and a positive test group (with the TTX concentration of 0.25 μg/L) tends to be stable after being gradually increased; and when the dilution fold of the TTX-BSA is 20, and the concentration of the TTX complete antigen (TTX-BSA) solution is 0.4 mg/mL, the competitive inhibition rate (1-T/T₀) between a negative sample and a positive sample reaches a maximal value of 0.3. Also, at this time, the detection line also has a comparatively higher fluorescence signal value of 343000 a.u. (the fluorescence intensity of the negative control: 499171 a.u.). Therefore, 20-fold dilution is a preferred dilution fold of the TTX-BSA complete antigen at the T line, and a concentration of 0.4 mg/mL is a preferred concentration of the TTX-BSA complete antigen at the T line.

(2) Optimization on quality control line of time-resolved immunoquantitation test strip

The biotin solution of the quality control line in embodiment 3 was adjusted to be a goat anti-mouse IgG solution; other kept consistent to those in embodiment 3; and the time-resolved immunoquantitation test strip was obtained.

The three samples confirmed to be negative by UPLC-MS/MS were labeled; the time-resolved immunoquantitation test strips with two kinds of quality control lines were selected for detecting positive samples with concentrations of 0.1 ng/mL, 0.5 ng/mL and 2 ng/mL (TTX) respectively; a mean concentration value, a standard deviation and a variable coefficient of the positive samples were calculated; and the accuracy and the precision of the time-resolved immunoquantitation test strip were evaluated.

TABLE 1
Experimental comparison on precision and stability of time-resolved
immunoquantitation test strips with different quality control lines

| | | | | | | | Meretrix | | | | |
|---|---|---|---|---|---|---|---|---|---|---|---|
| | | Labeling | | | | | meretrix | | | | |
| Quality | | concen- | Astarte | | | | linnaeus | | Clam | | |
| control | Target | tration | Mean | Recovery | CV | Mean | Recovery | CV | Mean | Recovery | CV |
| line | object | (ng/mL) | (ng/mL) | rate (%) | (%) | (ng/mL) | rate (%) | (%) | (ng/mL) | rate (%) | (%) |
| biotin- | TTX | 0.1 | 0.096 | 100.52 | 3.66 | 0.101 | 99.90 | 1.90 | 0.104 | 99.51 | 3.27 |
| BSA | | 0.5 | 0.460 | 101.48 | 4.70 | 0.492 | 100.29 | 2.90 | 0.544 | 98.49 | 3.13 |
| | | 2 | 2.266 | 97.08 | 3.21 | 1.848 | 101.86 | 0.25 | 2.143 | 98.39 | 3.39 |
| Goat | | 0.1 | 0.116 | 115.96 | 1.02 | 0.149 | 148.74 | 5.96 | 0.129 | 129.35 | 1.88 |
| anti- | | 0.5 | 0.578 | 115.63 | 7.06 | 0.552 | 110.34 | 11.97 | 0.753 | 150.53 | 18.26 |
| mouse | | 2 | 1.917 | 95.84 | 14.63 | 2.135 | 106.73 | 78.65 | 1.949 | 97.46 | 24.33 |
| IgG | | | | | | | | | | | |

Results are shown in Table 1. Determination results CV of the quality control line being biotin-BSA are all smaller than 4.70%, and the recovery rates are 97.08%-101.48%. However, the recovery rate of a traditional test strip with a goat anti-mouse IgG quality control line is large in fluctuation range and between 95.84% and 150.53%.

(3) Optimization on chromatography time

A chromatography time of step (3) in embodiment 5 was adjusted to be 0-20 min, and other kept consistent to those in embodiment 5.

With the fluorescence intensity values as ordinates and reaction times as abscissas, the immunoreaction kinetic curve was drawn, changes of the fluorescence intensities of the T line and the C line over time were observed, and the time that the fluorescence value of the T line became stable served as a preferred detection time.

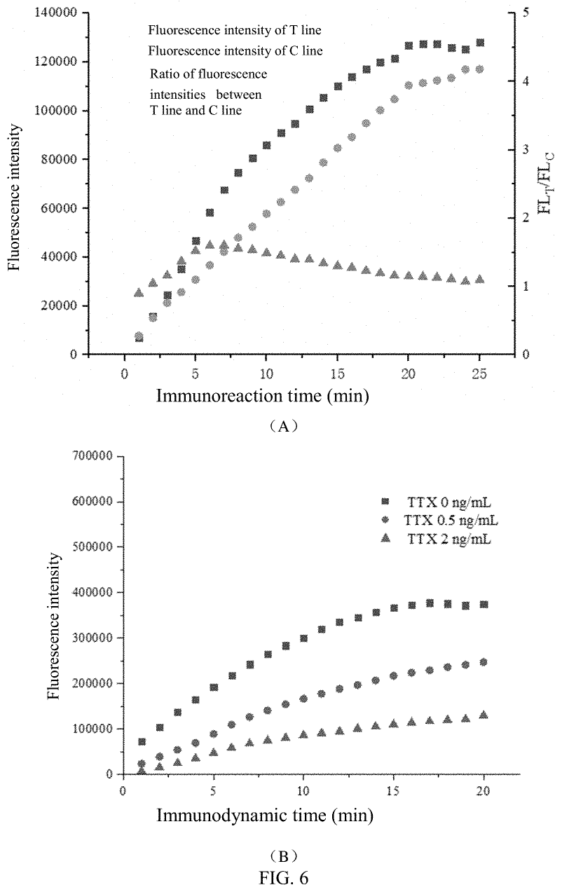

FIG. 6 is a diagram showing T line immunodynamic curves of TTX immunochromatographic assay. From A in FIG. 6, the fluorescence intensities of the two lines both present a strengthening trend within 0-20 min over time. After 20 min, the fluorescence intensity values of the T line and the C line have no obvious changes and trend to be stable. From B in FIG. 6, the fluorescence intensity between the negative control group (5% methanol-PBA) and the positive control group (TTX=0.5 μg/L, 2 μg/L) trends to be stable at 20 min Therefore, it is relatively proper to select 20 min as the chromatography time.

### Embodiment 7

1. Precision test

8 TTX time-resolved immunoquantitation test strips were selected from one batch for detecting fluorescence intensity values (To) at detection lines of a negative control group (5% methanol-PBA) and fluorescence intensity values (T) at T lines of a positive control group (TTX 0.25 μg/L), and intra-batch differences were analyzed.

8 TTX time-resolved immunoquantitation test strips in different batches were used for detecting the fluorescence intensity values (To) at the detection lines of the negative control group (5% methanol-PBA) and the fluorescence intensity values (T) at the T lines of the positive control group (TTX 0.25 μg/L), and inter-batch differences were analyzed.

From Table 2, through analysis with a variable coefficient computational formula, intra-batch variable coefficients of T₀, T and T/To (%) are 7.61%, 7.51% and 2.37% respectively, and inter-batch variable coefficients of T₀, T and T/T₀ (%) are 6.53%, 6.90% and 2.81% respectively, indicating that the time-resolved immunoquantitation test strips are small in intra-batch variable coefficients and inter-batch variable coefficients, high in precision and good in accuracy and basically meet the requirements of the quantitative detection test strip.

TABLE 2
Detection results of precision of time-resolved immunoquantitation test strip for TTX

| Intra- | | | T/T₀ | Inter- | | | T/T₀ | SD |
|---|---|---|---|---|---|---|---|---|
| batch | T₀ | T | (%) | batch | T₀ | T | (%) | (%) |
| 1 | 398312 | 318000 | 79.84% | 1 | 465546 | 372218 | 79.95% | 1.15% |
| 2 | 428656 | 330281 | 77.05% | 2 | 426906 | 321875 | 75.40% | 1.12% |
| 3 | 458968 | 346031 | 75.39% | 3 | 390765 | 296437 | 75.86% | 0.60% |
| 4 | 465875 | 346187 | 74.31% | 4 | 449250 | 341484 | 76.01% | 0.58% |
| 5 | 464765 | 365656 | 78.68% | 5 | 449250 | 337875 | 75.21% | 3.19% |
| 6 | 455546 | 350687 | 76.98% | 6 | 458125 | 348968 | 76.17% | 0.57% |
| 7 | 453734 | 350078 | 77.15% | 7 | 470843 | 340468 | 72.31% | 2.89% |
| 8 | 377812 | 284812 | 75.38% | 8 | 407343 | 314859 | 77.30% | 1.43% |
| Mean | 437959 | 336467 | 76.85% | Mean | 439754 | 334273 | 76.03% | |
| SD | 33324 | 25278 | 1.82% | SD | 28698 | 23057 | 2.14% | |
| CV (%) | 7.61% | 7.51% | 2.37% | CV (%) | 6.53% | 6.90% | 2.81% | |

2. Accuracy test

Sample Treatment:

With regard to a sample pre-treatment mode of shellfish products, 5 g of shellfish meat was collected, 25 mL of 0.1% glacial acetic acid was added for homogenizing, and a water bath was performed at 100° C. for 10 min after homogenizing; standing was performed for cooling, centrifugation was performed at 10,000 r/min to collect a supernate, an appropriate amount of 0.1% glacial acetic acid was added into precipitates for uniform mixing, and a water bath was performed for 5 min; standing was performed for cooling, centrifugation was performed at 10,000 r/min for 5 min, a supernate was collected, and the above step was repeated for precipitates once; the three supernates were collected to a constant volume of 50 mL; then, 5 mL of chloroform was added for degreasing for 30 min, during which shaking was performed for uniform mixing 5 times, centrifugation was performed at 10,000 r/min for 3 min to separate an aqueous phase and chloroform, an aqueous phase was collected, degreasing was performed once again, and a pH value of the aqueous phase was adjusted to 7.0 with NaOH, so as to obtain the to-be-detected substance; and if not being detected directly, the to-be-detected substance might be preserved at 4° C. (without adjusting the pH value) (GB/T5009).

In order to verify the accuracy and the sensitivity of the TTX test strip, labeled recovery tests are performed on several kinds of shellfish samples (astarte, clams and Meretrix meretrix linnaeus); addition concentrations of each sample are set to be there different labeling concentrations in a high group, a medium group and a low group; and for each group of concentration gradient, three parallel tests are set. With the labeled recovery rate as an accuracy evaluation index, a relative standard deviation (RSD %) of a detection result of a certain concentration of sample is repeatedly determined as a precision evaluation index. Computational formulas of the labeled recovery rate and the relative standard deviation are shown as following formula (1) and formula (2):

$$Labeled recover rate ( \% ) = \\ \frac{Actually detected concentration of labeled sample}{Labeling concentration} ⨯ 100 \qquad ( 1 )$$

$$Variable coefficient ( CV , \% ) = \frac{Standard deviation ( SD )}{Mean} \qquad ( 2 )$$

TTX labeled recovery tests were performed on the samples confirmed to be negative according to three concentration gradients of 0.1 μg/L, 0.5 μg/L and 2 μg/L respectively. The test for each concentration was repeated for 10 times, a mean of T/To was taken, and a variable coefficient was calculated.

Results are shown in Table 3, the determination results CV are all smaller than 4.70%, which shows that in a linearity range, the time-resolved immunoquantitation test strip has relatively good accuracy. The whole exploration method takes the PBS containing 0.05% Tween-20 as a sample diluent, so as to guarantee the performance of the method.

5 g of each of the astarte, the clams and the Meretrix meretrix linnaeus was collected, 25 mL of 0.1% glacial acetic acid was added to each for homogenizing, and a water bath was performed at 100° C. for 10 min after homogenizing; standing was performed for cooling, centrifugation was performed at 10,000 r/min for 5 min, a supernate was collected, an appropriate amount of 0.1% glacial acetic acid was added into precipitates for uniform mixing, and a water bath was performed for 5 min; standing was performed for cooling, centrifugation was performed at 10,000 r/min for 5 min, a supernate was collected, and the above step was repeated for precipitates once; the three supernates were collected to a constant volume of 50 mL; then, 5 mL of chloroform was added for degreasing for 30 min, during which shaking was performed for uniform mixing 5 times, centrifugation was performed at 10,000 r/min for 3 min to separate an aqueous phase and chloroform, an aqueous phase was collected, degreasing was performed once again, and a pH value of the aqueous phase was adjusted to 7.0 with NaOH; and resultants were diluted by 15 folds (astarte), 5 folds (clam) and 5 folds (Meretrix meretrix linnaeus) respectively for immunochromatographic assay.

TABLE 3
Detection results of accuracy of time-resolved
immunoquantitation test strip for TTX

| | Labeling | Immunochromatographic test strip | | | |
|---|---|---|---|---|---|
| | concentration | Tested | Actual | Recovery | CV |
| Sample | (ng/mL) | T/T₀ (%) | T/T₀ (%) | rate (%) | (%) |
| Astarte | 0.1 | 0.840450 | 0.844863 | 100.53% | 3.66% |
| | 0.5 | 0.653965 | 0.663652 | 101.48% | 4.70% |
| | 2 | 0.493335 | 0.478888 | 97.07% | 3.21% |
| Meretrix | 0.1 | 0.840450 | 0.839679 | 99.91% | 1.90% |
| meretrix | 0.5 | 0.653965 | 0.655895 | 100.30% | 2.90% |
| linnaeus | 2 | 0.493335 | 0.502486 | 101.85% | 0.25% |
| Clam | 0.1 | 0.840450 | 0.836378 | 99.52% | 3.27% |
| | 0.5 | 0.653965 | 0.644125 | 98.50% | 3.13% |
| | 2 | 0.493335 | 0.485355 | 98.38% | 3.39% |

From Table 3, by using the time-resolved immunoquantitation test strip for detecting the labeled shellfish samples, it is finally obtained that, for the shellfishes, the labeled recovery rate is between 97.07% and 101.85%, and the variable coefficient is lower than 4.70%, indicating that the time-resolved immunoquantitation test strip can be used for field rapid screening and detection.

3. Standard curve for shellfish foods

In order to further guarantee the accuracy of the method in embodiment 5, with samples confirmed to be negative by LC-MS as a substrate, the TTX standard substance was added to the shellfish meat to prepare shellfish samples containing 0 ng/mL, 0.1 ng/mL TTX, 1 ng/mL TTX, 2 ng/mL TTX, 4 ng/mL TTX, 8 ng/mL TTX, 20 ng/mL TTX, 40 ng/mL TTX, 80 ng/mL TTX and 100 ng/mL TTX respectively, and a shellfish meat standard curve which was added to the shellfish meat to prepare shellfishsuitable for field immediate detection was drawn.

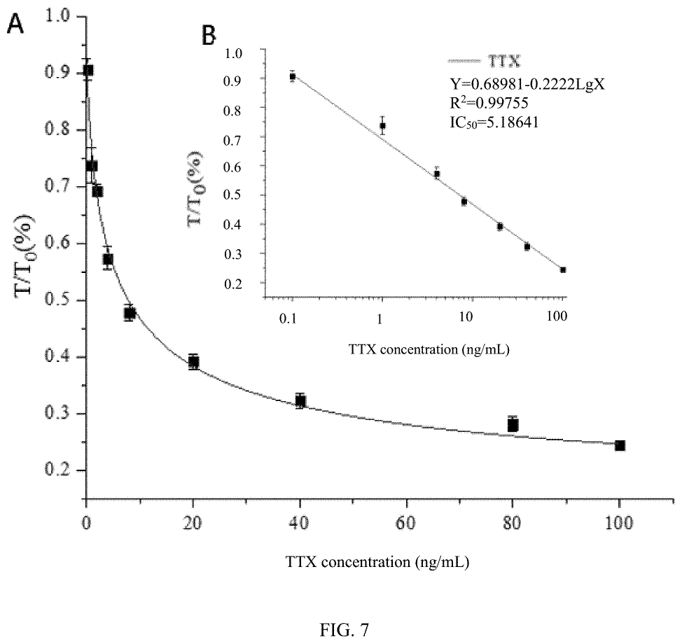

Results are shown in FIG. 7, when a concentration range of the TTX is from 0.1 ng/mL to 100 ng/mL, a logarithmic value of the concentration has a linear relationship with T/To; a linear equation is: Y=−0.22LgX+0.690, and R²=0.9976; and a limit of detection is 0.141 ng/mL.

4. Cross reaction test

Other commonly used shellfish toxins (OA, DA, BTX, ATX and MC-LR) were selected for evaluating the specificity of the time-resolved immunoquantitation test strip.

The negative samples (5% methanol-PBS) and positive samples with different concentrations (0 ng/mL, 0.5 ng/mL, 1.25 ng/mL, 2.5 ng/mL, 5 ng/mL, 10 ng/mL and 50 ng/mL) of each toxin were detected by using the time-resolved immunoquantitation test strips in embodiment 3, and the fluorescence intensity values of the T lines thereof were recorded.

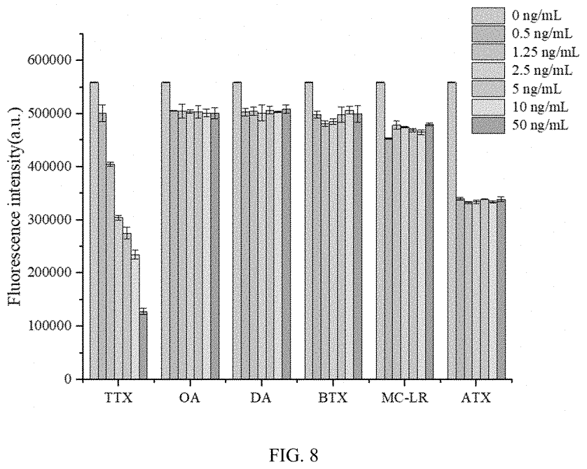

From FIG. 8 and Table 4, cross reaction between the TTX and other shellfish toxins is relatively high, which shows that the time-resolved immunoquantitation test strip is stable in performance, meets the market demands and has a wide market prospect.

TABLE 4
Results of cross reaction test

| Shellfish | FL_{T} (a.u.) | | | | | |
|---|---|---|---|---|---|---|
| toxins | 0.5 ng/mL | 1.25 ng/mL | 2.5 ng/mL | 5 ng/mL | 10 ng/mL | 50 ng/mL |
| TTX | 501250 | 404713 | 304286 | 275250 | 234760 | 128011 |
| OA | 505619 | 505200 | 504166 | 503437 | 501617 | 501086 |
| DA | 503422 | 504729 | 501687 | 506770 | 503922 | 508625 |
| BTX | 498656 | 481594 | 485766 | 498521 | 506703 | 500083 |
| MC-LR | 452864 | 478692 | 475179 | 469265 | 465390 | 480375 |
| ATX | 339807 | 333160 | 334197 | 338692 | 333505 | 339010 |

Although the present invention has been disclosed as above in the preferred embodiments, they are not intended to limit the present invention. Those skilled in the art can make various changes and modifications without departing from the spirit and scope of the present invention. Therefore, the protection scope of the present invention is subject to the protection scope in claims.

What is claimed is:

1. A time-resolved immunochromatography quantitative test strip for detecting tetrodotoxin (TTX), comprising a sample pad, a nitrocellulose membrane (an NC membrane) and absorbent paper, wherein a detection line (a T line) and a quality control line (a C line) are arranged on the NC membrane; a TTX complete antigen (TTX-BSA) is coated on the detection line; and a biotin (biotin-BSA) is coated on the quality control line, wherein the dosage of the TTX complete antigen is 0.3-8 mg/mL, and the dosage of the biotin is 0.1-1 mg/mL.

2. The time-resolved immunochromatography quantitative test strip according to claim 1, wherein the detection concentration of the TTX is 0.1-1000 ng/mL.

3. The time-resolved immunochromatography quantitative test strip according to claim 1, wherein in a preparation method of the TTX complete antigen (TTX-BSA), the TTX complete antigen (TTX-BSA) is obtained by coupling a TTX standard substance and a carrier protein.

4. A method of detecting TTX in shellfishes by a time-resolved immunoquantitation test strip, comprising the following steps:
(1) extraction of a to-be-detected substance: homogenizing shellfish meat, heating, cooling, centrifuging, and collecting supernate; and then, degreasing to obtain the to-be-detected substance;
(2) uniformly mixing a Eu³⁺-fluorescent microsphere labeled TTX monoclonal antibody Eu-TTX-mAb, a Eu³⁺-fluorescent microsphere labeled streptavidin Eu-SA, the to-be-detected substance and buffer solution for hatching, so as to obtain to-be-detected solution;
(3) adding the to-be-detected solution obtained in step (2) into the sample pad in the time-resolved immunochromatography quantitative test strip described in the present invention, and then determining the fluorescence intensity value at the detection line of a sample: a T value; and
(4) substituting the obtained T value into a quantitative curve, so as to obtain the concentration of the TTX in the to-be-detected solution.

5. The method according to claim 4, wherein the volume ratio of the Eu³⁺-fluorescent microsphere labeled TTX monoclonal antibody Eu-TTX-mAb, the Eu³⁺-fluorescent microsphere labeled streptavidin Eu-SA, the to-be-detected substance and the buffer solution in step (2) is 2:10:45:35.

6. The method according to claim 4, wherein the hatching in step (2) is carried out at 20-30° C. for 10-20 min.

7. The method according to claim 4, wherein the chromatography in step (3) is carried out at 37° C. for 15-20 min.

8. The method according to claim 4, wherein the quantitative curve in step (4) is Y=0.57365-0.2668LgX, R²=0.9940, IC50=1.713 ng/mL, and the limit of detection is 0.047 ng/mL, wherein Y represents T/To, and X represents the concentration of the TTX.

9. An application of the time-resolved immunochromatography quantitative test strip of claim 1 in the field of food detection.

10. An application of the method of claim 4 in the field of food detection.
